(19) United States
(12) Patent Application Publication
(10) Pub. No.: US 2026/0316334 A1
(43) Pub. Date: Oct. 15, 2026

(54) SYSTEM AND METHOD FOR DETERMINING MINIMUM PASTE ADDITION

(71) Applicant: X DEVELOPMENT LLC, Mountain View, CA (US)

(72) Inventor: Weishi Yan, Oakland, CA (US)

(21) Appl. No.: 19/482,059

(22) PCT Filed: Jun. 20, 2024
(86) PCT No.: PCT/US2024/034766

§ 371 Date: Nov. 6, 2025

Related U.S. Application Data
(60) Provisional application No. 63/509,473, filed on Jun. 21, 2023.

(51) Int. Cl. B28C 7/02; B28C 7/04; C04B 40/00; C04B 103/00; G05B 17/02

(52) U.S. Cl. CPC B28C 7/024; B28C 7/0418; C04B 40/0032; G05B 17/02; C04B 2103/0039

(57) ABSTRACT

$${v}_{i}^{p} \qquad ( I )$$

$${s}_{i}^{p} \qquad ( II )$$

$${a}_{i}^{p} \qquad ( III )$$

$${v}_{i}^{s} \qquad ( IV )$$

$${s}_{i}^{s} \qquad ( V )$$

This disclosure involves a system, non-transitory computer readable medium, and a method for determining a minimum amount of paste to be added to a concrete mix. This includes measuring a plurality of n particles, and for each particle i determining: a particle volume (I), a particle surface area (II), a particle maximum dimension (III), a spherical volume (IV) and spherical surface area (V) associated with a sphere having a diameter equal to the particle maximum dimension (III), and a residual free surface Sᵢ. Then, for the plurality of particles, a shape irregularity factor η is determined based on the residual free surface Sᵢ and the particle volume (I) for all n particles. The shape irregularity factor is used to determine a minimum amount of paste for combination with the plurality of n particles to create a workable concrete mixture, and a control signal is sent to a concrete preparation system.

### BACKGROUND

Concrete is the second most consumed substance (by mass) on our planet and is responsible for 7-8% of global CO2 emissions. Concrete's material properties are inconsistent due to the large variation in ingredient material (e.g., aggregates) and processing. This material inconsistency requires large safety margins for a given performance level and results in material overuse. Advances in concrete preparation that can optimize the use of locally available materials to maximize concrete performance while minimizing cost with both traditional and non-traditional concrete ingredients are desirable.

### SUMMARY

In general, the disclosure involves a system, non-transitory computer readable medium, and a method for determining a minimum amount of paste to be added to a concrete mix. This includes measuring a plurality of n particles, and for each particle i determining: a particle volume

$${v}_{i}^{p} ,$$

a particle surface area

$${s}_{i}^{p} ,$$

a particle maximum dimension

$${a}_{i}^{p} ,$$

a spherical volume

$${v}_{i}^{s}$$

and spherical surface area

$${s}_{i}^{s}$$

associated with a sphere having a diameter equal to the particle maximum dimension

$${a}_{i}^{p} ,$$

and a residual lice surface Sᵢ. Then, for the plurality of particles, a shape irregularity factor η is determined based on the residual free surface Sᵢ and the particle volume

$${v}_{i}^{p}$$

for all n particles. The shape irregularity factor is used to determine a minimum amount of paste for combination with the plurality of n particles to create a workable concrete mixture, and a control signal is sent to a concrete preparation system to cause the concrete preparation system to add at least the minimum amount of paste to a concrete mix that includes the plurality of n particles.

Implementations can optionally include one or more of the following features.

In some instances, determining the residual free surface Sᵢ for each particle i includes calculating

$$( \frac{{s}_{i}^{p}}{{v}_{i}^{p}} - \frac{{s}_{i}^{s}}{{v}_{i}^{s}} ) {v}_{i}^{p} .$$

In some instances, determining the shape irregularity factor η includes calculating

$$\frac{4 π}{3 {( 4 π )}^{\frac{3}{2}}} \frac{{\sum }_{i}^{n} {s}_{i}^{\frac{3}{2}}}{{\sum }_{i}^{n} {v}_{i}^{p}}$$

for all n particles.

In some instances, determining the minimum amount of paste for combination with the plurality of n particles comprises calculating (1−ρ)+ρη, where ρ is a packing density of the plurality of n particles. In some instances, the packing density ρ is a ratio of volume occupied by the plurality of n particles to the volume of the n particles.

In some instances, determining the minimum amount of paste to combine with the plurality of n particles includes applying a correction factor to the shape irregularity factor, the correction factor based on a difference in density between the plurality of n particles and the paste.

In some instances, measuring the plurality of n particles includes scanning each particle with an optical scanner and generating a three-dimensional mesh model for each particle.

In some instances, the particle maximum dimension represents the longest straight line length of the particle i.

In some instances, the workable concrete mixture is a concrete mixture with a slump in a range of two to eight inches, wherein slump is measured according to ASTM C143.

In some instances, sending the control signal to the concrete preparation system includes controlling operation of a conveying mechanism to add a volume of paste greater than or equal to the minimum amount, to a mixing vessel of the concrete preparation system.

The details of one or more implementations of the subject matter of this specification are set forth in the accompanying drawings and the description below. Other features, aspects, and advantages of the subject matter will become apparent from the description, the drawings, and the claims.

### DESCRIPTION OF DRAWINGS

This disclosure relates to determining a minimum required paste to achieve workable concrete for a given aggregate.

FIG. 1 depicts an exemplary concrete preparation system.

FIG. 2 is a block diagram of an exemplary control system.

FIG. 3 is a flow diagram illustrating an example process for sending control signals to a concrete preparation system.

FIG. 4 is a schematic diagram of a computer system.

### DETAILED DESCRIPTION

This disclosure describes a system and method for determining and adding an amount of cement paste to an aggregate in order to achieve a desired workability in a concrete mixture. Concrete includes a mixture of various ingredients, many of which include particulates of varying sizes and shapes. Freshly mixed concrete needs to be workable, or able to mold or form into the desired final shape prior to curing. In other words, the workability of the concrete determines how useable it is for operations such as pumping, pouring, spreading, tamping or flowing into a mold or fixture. Non-spherical particles within an aggregate can impart shear forces on one another, resulting in a mixture that doesn't “flow” or isn't workable. One way to improve the workability is to increase the amount of small particles such as cement paste within the mixture such that the large aggregate particle interactions are minimized. However, cement paste is relatively expensive and heavy, so it is often desirable to include a minimum amount of cement paste required to coat the aggregate and achieve the workability desired.

By measuring and modeling each large particle of the aggregate, an approximation of how “rough” or “jagged” a group of particles forming an aggregate is, which can in turn be used to calculate a minimum paste amount to achieve a desired workability. In some implementations, concrete's workability is measured using a slump test, ASTM C143. In ASTM C143, a cone is filled with concrete and tamped according to a particular procedure, then the cone is removed, and as the concrete relaxes, a height difference is measured in inches to determine slump. In some implementations, a desirable slump is between 2″ and 8″ when measured using ASTM C143.

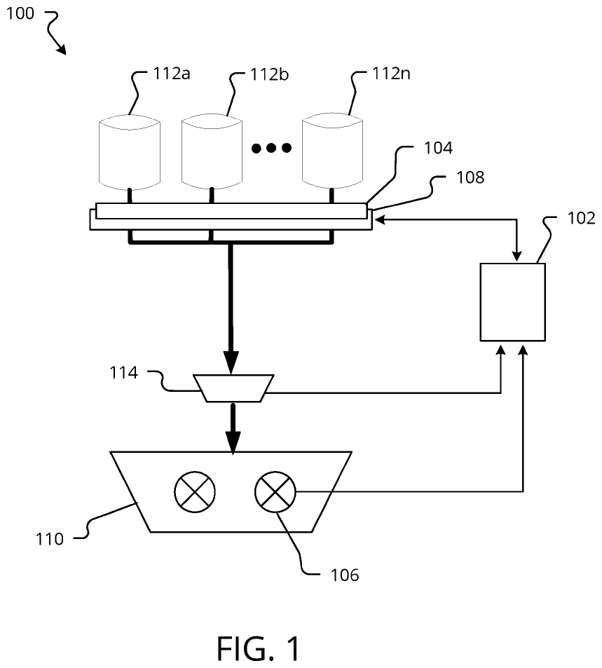

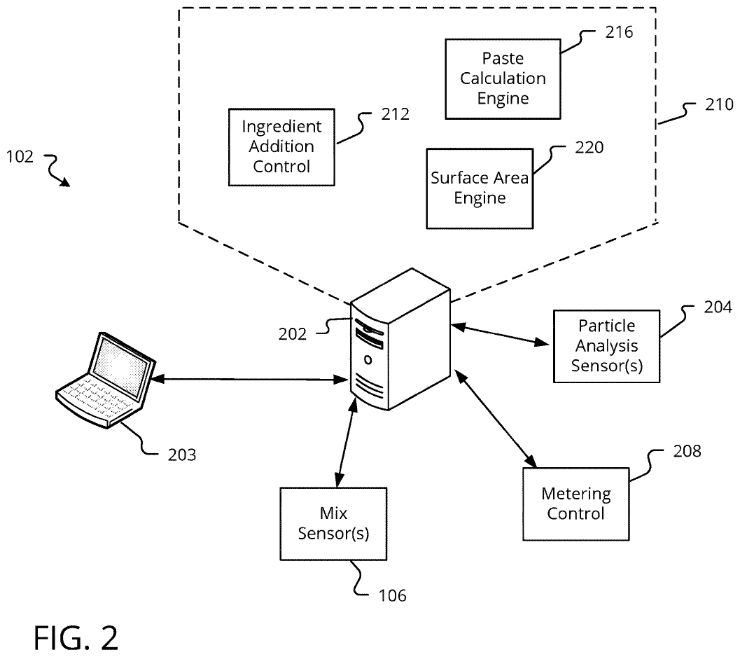

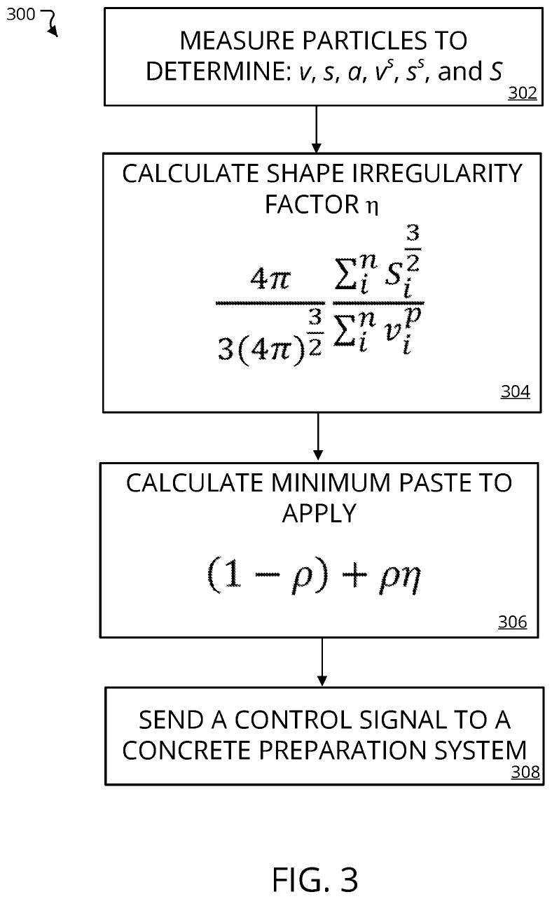

FIG. 1 depicts an exemplary concrete preparation system 100. In operation, concrete preparation system 100 measures characteristics of the raw ingredients of a concrete mixture. Concrete preparation system 100 can adaptively adjust the proportion of the raw ingredients added to the concrete mixture based on the measured or predicted characteristics of raw ingredients to more accurately achieve desired structural properties in the final cured concrete. The operation of the concrete preparation system 100 is described in more detail below in reference to FIGS. 2 and 3.

Concrete preparation system 100 includes a control system 102. The control system 102 receives input from particle analyzing system 104 and concrete mix sensors 106. The control system 102 can control the operations of one or more ingredient metering systems 108 based on analyses of data obtained from one or both of the particle analyzing system 104 and concrete mix sensors 106.

Concrete preparation system 100 includes raw ingredient storage bays or hoppers 112a-112n. The ingredient metering system 108 conveys the raw ingredients from the storage bays 112a-112n to a mixing vessel 110. For example, the ingredient metering system 108 can include a series of conveyors and augers to transfer raw ingredients from the storage bays 112a-112n into the mixing vessel 110. In some implementations, raw ingredients include various aggregates such as crushed stone, sand, gravel or a combination thereof, cement mixture, water, admixtures, fly ash, metal densifiers, or other ingredients. In some implementations storage bays 112a store coarse aggregates, which have a particle size that is ten to one hundred times larger than fine aggregates, which themselves have particle sizes greater than the particle size of a cement paste (e.g., mixture of cement and water).

In some implementations, the ingredient metering system 108 may include a metering hopper 114 between the ingredient metering system 108 and the mixing vessel 110. The metering hopper 114 may be used to collect and measure (e.g., weigh) a raw ingredient before it enters mixing vessel 110. For example, the weight of the ingredient measured by metering hopper 114 can be passed to the control system 102 permitting the control system to monitor the weight of the ingredient being measured in real-time. The control system 102 may then be able to make in-situ adjustments to how much of the ingredient to add to the concrete mixture based on real-time particle analysis of the ingredient from the particle analyzing system 104. In some implementations, concrete preparation system 100 can be retro-fit to a traditional ready-mix concrete plant. For example, adding the concrete preparation system 100 to a ready-mix plant may allow the ready-mix plant to more precisely tailor concrete mixes for specific applications and job sites.

The particle analyzing system 104 measures each particle that exits storage bays 112a-112n. Some implementations may include a series of sieves to separate particles of an ingredient by size. In such implementations, the optical sensors (e.g. within the particle analyzing system 104) can be positioned proximate to each sieve to capture images of the particles passing through the sieve. The images can then be used, for example, with a computer imaging system to generate a three-dimensional mesh model for each particle at a particular gradation in the series of sieves. The three-dimensional mesh model can include a wireframe geometry that represents the particle, and can be used to determine geometric features of each particle such as maximum dimensions along each of three orthogonal axes, as well as particle surface area. In some implementations, the separated particles may be recombined before being added to the mixing vessel 110. In some implementations, the optical sensors are positioned to capture images of particles as they pass on a conveyor from one sieve to a final mixer, e.g., mixing vessel 110.

The concrete mix sensors 106 provide rheometry measurements of the concrete mixture to the control system 102. For example, the concrete mix sensors 106 can measure various attributes of the concrete mixture that can be used to estimate or compute rheumatic properties of the concrete mixture in real-time. The concrete mix sensors 106 can include, but are not limited to, viscosity sensors, rheometers, temperature sensors, moisture sensors, ultrasonic sensors (e.g., ultrasonic pulse velocity sensors), electrical property sensors (e.g., electrodes, electrical resistance probes), electromagnetic sensors (e.g., short-pulse radar), or other sensors (e.g., geophone, accelerometer). The concrete mix sensors 106 can include, but are not limited to, hydrophobicity, moisture content, XRD spectra, XRF spectra, static yield stress, acoustic impedance, p-wave speed, dynamic yield stress, static modulus of elasticity, Young's modulus, bulk modulus, shear modulus, dynamic modulus of elasticity (DME), Poisson's ratio, density, resonance frequency, nuclear magnetic resonance (NMR), dielectric constant, electric resistivity, polarization potential, and capacitance.

For example, viscosity, moisture, and temperature sensors can be installed in the mixing vessel 110. These sensors can be used to measure rheologic properties of the concrete mixture such as changes in the viscosity of the mixture over time and at different moisture content levels and temperatures. As described in more detail below, the control system 102 can use the rheometry measurements to determine whether and how much additional ingredients should be added to the concrete mixture to obtain desired concrete properties.

FIG. 2 is a block diagram of an exemplary control system 102 for the concrete preparation system 100. The control system 102 includes a computing system 202 in communication with the concrete mix sensors 106, particle analysis sensors 204, a metering control system 208 which can control operations of the ingredient metering system 108. Computing system 202 is configured to control various aspects of the concrete preparation process. For example, computing system 202 can store and execute one or more computer instruction sets to control the execution of aspects of the concrete preparation processes described herein. Computing system 202 can include a system of one or more computing devices. The computing devices can be, e.g., a system of one more servers. For example, a first server can be configured to receive and process data from the concrete mix sensors 106 and the particle analysis sensors 204. Another server can be configured to interface with the metering control system 208 and issue control commands based on analysis results from the first server.

In some implementations, the computing system 202 can be operated or controlled from a user computing device 203. User computing device 203 can be a computing device, e.g., desktop computer, laptop computer, tablet computer, or other portable or stationary computing device.

Briefly, computing system 202 can control the overall concrete preparation system 100 to prepare concrete mixtures. The computing system 202 can use the particle analysis sensors 204 to characterize concrete ingredients as they are added to a concrete mixture. The computing system 202 obtains rheometry measurements from the mix sensors 106 as the concrete mixture is mixed in the mixing vessel 110. The system compares the rheometry measurements with estimated rheometry measurements to determine, e.g., whether the concrete mixture will meet desired post-curing mechanical properties or whether additional ingredients should be added.

In some implementations, computing system 202 can include a set of operations modules 210 for controlling different aspects of a concrete additive manufacturing process. The operation modules 210 can be provided as one or more computer executable software modules, hardware modules, or a combination thereof. For example, one or more of the operation modules 210 can be implemented as blocks of software code with instructions that cause one or more processors of the computing system 202 to execute operations described herein. In addition, or alternatively, one or more of the operations modules can be implemented in electronic circuitry such as, e.g., programmable logic circuits, field programmable logic arrays (FPGA), or application specific integrated circuits (ASIC). The operation modules 210 can include an ingredient addition controller 212, paste calculation engine 216, and surface area engine 220.

Ingredient addition controller 212 interfaces with the metering control system 208 to control the addition of ingredients to the concrete mixing vessel 110. For example, the ingredient addition controller 212 can issue commands from the computing system 202 to the metering control system 208 to control the addition of ingredients to the concrete mixture in the mixing vessel 110 by increasing a conveyor or auger speed, opening or closing a sluice, or otherwise change the addition rate of one or more ingredients.

Surface area engine 220 can receive measured parameters associated with aggregate or other large particles and determine a surface area for each particle. For example, surface area engine 220 can receive a three-dimensional mesh model for N large particles. In some implementations, the three-dimensional mesh model will be formed of a number of vertices which define a number of triangles. For each mesh model, the surface area engine 220 can sum an area of all of the triangles within the mesh to calculate a total surface area. For example, given an arbitrary triangle defined by points A, B, and C, the area of that triangle will be equal to

$$\frac{1}{2} \vert A \times B + B \times C + C \times A \vert$$

(where x denotes a cross product). In some implementations, surface area engine 220 receives different parameters for each particle. Alternatively, the area of the triangle can be calculated using Heron's formula: AREA=\sqrt{s(s−A)(s−B)(s−C)} where

$$s = \frac{A + B + C}{2}$$

For example, in some implementations the surface area engine 220 receives three maximum or principal lengths along three orthogonal axis for each particle. In these implementations, the surface area engine 220 can assume the particle is an ellipsoid in shape, and using the three lengths, calculate the surface area of that ellipsoid. In some instance, the surface area engine 220 can add an additional correction factor, to compensate for the difference between the assumed ellipsoid and the actual particle shape. In addition to surface area, the surface area engine 220 can calculate particle volume for each particle. The volume can be calculated based on a mesh model, or height map image, or other techniques. Given the particle volume, the principal lengths, and surface area, the surface area engine 220 can then calculate a residual free surface area, which represents the excessive surface area of the particle due to particle convexity, angularity, and/or non-sphericity. In other words, the residual free surface area for a particle represents how much surface area the particle has compared to an ideal spherical shape that encloses the particle.

The paste calculation engine 216 can determine, based on the volume and residual free surface area for each particle as determined by surface area engine 220, an amount of paste that needs to be added for a given group of large particles (e.g., aggregate). This amount of paste, sometimes referred to as a free paste demand, or shape irregularity factor, can be determined by summing the free residual surface area for each particle in the group of large particles, risen to the power of 3/2 and normalized by a sum of particle volumes, then multiplied by a constant C of

$$\frac{4 π}{3 {( 4 π )}^{\frac{3}{2}}} .$$

This calculation determines a free paste demand, or shape irregularity factor η. The shape irregularity factor can be used to determine a minimum amount of paste required by calculating (1−ρ)+ρη, where ρ is the packing density for the group of large particle. Packing density can be determined by dividing the total volume occupied by the group of large particles by the sum of the volume for each individual particle in the group of large particles.

FIG. 3 is a flow diagram illustrating an example process 300 for sending control signals to a concrete preparation system. It will be understood that process 300 may be performed, for example, by any suitable system, environment, software, and hardware, or a combination of systems, environments, software, and hardware as appropriate. In some instances, process 300 can be performed by the system as described in FIG. 1, or portions thereof, and further described in FIG. 2, as well as other components or functionality described in other portions of this description. In other instances, process 300 may be performed by a plurality of connected components or systems. Any suitable system(s), architecture(s), or application(s) can be used to perform the illustrated operations.

At 302, a group of particles is measured in order to determine, for each particle, its volume v^{p}, surface area s^{p}, longest dimension a^{p}, spherical volume v^{s}, spherical surface area s^{s}, and residual free surface area S. These measurements can be taken for each particle in the group of particles, for example, using cameras and photogrammetry as the particles pass by on a conveyor system of fall through a sensing system (e.g., metering system 108 of FIG. 1). The spherical volume v^{s} and spherical surface s^{s} area are based on a sphere that encloses the particle entirely, or a sphere with a diameter equal to the measured longest dimensions a^{p}. The residual free surface area S compares the surface area measured of the particle to the spherical surface area, and can be found using the equation

$${S}_{i} = ( \frac{{s}_{i}^{p}}{{v}_{i}^{p}} - \frac{{s}_{i}^{s}}{{v}_{i}^{s}} ) {v}_{i}^{p} ,$$

where i is an index representing the particular particle. In some implementations, every particle in a group of particles is measured. In some implementations, where accurate sampling is possible, a sample, or subset of the total group of particles is measured, and the parameters are approximated for the remaining particles in the group.

At 304, a shape irregularity factor or residual free surface n is determined. The shape irregularity factor η can be determined based on the residual free surface area for every particle in the group of particles, and the volume of those particles, and can be calculated according to the equation

$$η = C \frac{{\sum }_{i}^{n} {S}_{i}^{\frac{3}{2}}}{{\sum }_{i}^{n} {v}_{i}^{p}} where C = \frac{4 π}{3 {( 4 π )}^{\frac{3}{2}}} .$$

Shape irregularity factor can represent how irregular the large particles in the group of particles are, as compared to a group of sphere shaped particles. The shape irregularity factor required for a given group of particles requires that the group of particles have similar density to the paste. For example, a typical aggregate mixture may include large particles that have a density of approximately 2.7 g/cm³, and the paste used may be approximately 2.4 g/cm³. In some implementations, an additional correction factor can be applied to the shape irregularity factor η in order to compensate for differing density between the paste and the aggregate. For example, in the foregoing example, a

$$\frac{2.7}{2.4} .$$

At 306, the shape irregularity factor η for the group of particles can be used to determine how much paste is needed to ensure large particles in the mixture will be sufficiently coated to minimize large particle interaction, yielding a workable mixture. This minimum amount of paste can be determined by calculating (1−ρ)+ρη, where ρ is a packing density of the group of particles. Packing density ρ can be calculated by dividing the total volume occupied by the group of particles, by the sum of their individual volumes. In some implementations, the packing density for typical concrete aggregate is in the range of 0.48 to 0.52. In calculating the minimum amount of paste, the term (1−ρ) represents the void volume, that will need to be filled with paste. The term ρη represents the amount of paste required to coat the large particles, reducing or eliminating shear forces between the large particles, and allowing the mixture to be workable. In some implementations workability is measured using a slump test, such as ASTM C143, and a mixture is considered workable where the mixture has a slump between 2″ and 8″.

In some implementations, 304 can be performed separately from 306. For example, during mining operations, aggregate can be created by extracting rock, crushing it, and passing it through a series of sieves to graduate it. The graduated aggregate can be analyzed at this time to determine the shape irregularity factor n. Then batches of aggregate (e.g., bags, trucks, containers, etc.) can be associated with an n value before they are delivered to a preparation facility, where 306 is performed.

At 308, a control signal is sent to a concrete preparation system to add at least the determined minimum amount of paste to a concrete mix that includes the group of particles. The control signal can activate or alter the operation of a delivery system that applies paste (e.g., cement paste) to the aggregate. For example, the control signal can open or close a valve. It can speed up or slow down an auger, or conveyor. In some implementations, the control signal can work to alter a pressure, for example, where the paste is delivered via an injection system or a pipe network that uses pneumatic or hydraulic pressure as a motive force.

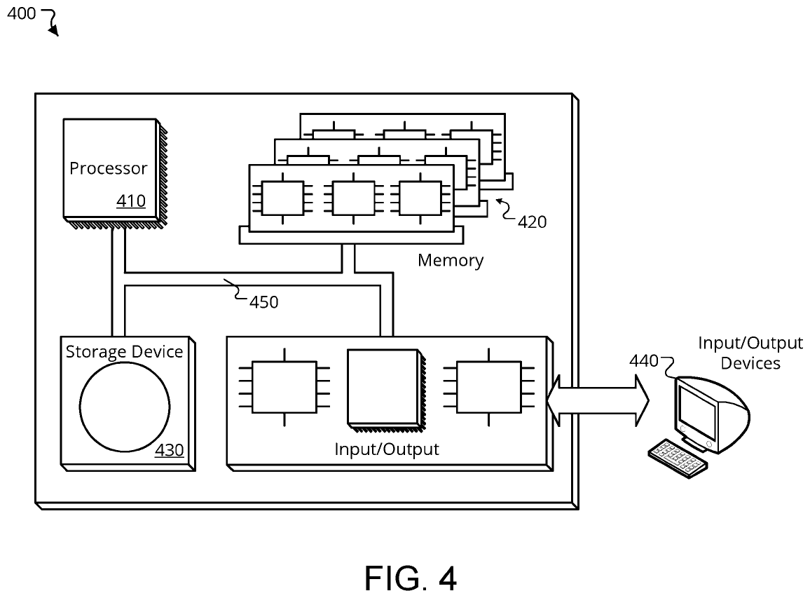

FIG. 4 is a schematic diagram of a computer system 400. The system 400 can be used to carry out the operations described in association with any of the computer-implemented methods described previously, according to some implementations. In some implementations, computing systems and devices and the functional operations described in this specification can be implemented in digital electronic circuitry, in tangibly embodied computer software or firmware, in computer hardware, including the structures disclosed in this specification and their structural equivalents, or in combinations of one or more of them. The system 400 is intended to include various forms of digital computers, such as laptops, desktops, workstations, servers, blade servers, mainframes, and other appropriate computers. The system 400 can also include mobile devices, such as personal digital assistants, cellular telephones, smartphones, and other similar computing devices. Additionally, the system can include portable storage media, such as Universal Serial Bus (USB) flash drives. For example, the USB flash drives may store operating systems and other applications. The USB flash drives can include input/output components, such as a wireless transducer or USB connector that may be inserted into a USB port of another computing device.

The system 400 includes a processor 410, a memory 420, a storage device 430, and an input/output device 440. Each of the components 410, 420, 430, and 440 are interconnected using a system bus 450. The processor 410 is capable of processing instructions for execution within the system 400. The processor may be designed using any of a number of architectures. For example, the processor 410 may be a CISC (Complex Instruction Set Computers) processor, a RISC (Reduced Instruction Set Computer) processor, or a MISC (Minimal Instruction Set Computer) processor.

In one implementation, the processor 410 is a single-threaded processor. In another implementation, the processor 410 is a multi-threaded processor. The processor 410 is capable of processing instructions stored in the memory 420 or on the storage device 430 to display graphical information for a user interface on the input/output device 440.

The memory 420 stores information within the system 400. In one implementation, the memory 420 is a computer-readable medium. In one implementation, the memory 420 is a volatile memory unit. In another implementation, the memory 420 is a non-volatile memory unit.

The storage device 430 is capable of providing mass storage for the system 400. In one implementation, the storage device 430 is a computer-readable medium. In various different implementations, the storage device 430 may be a floppy disk device, a hard disk device, an optical disk device, or a tape device.

The input/output device 440 provides input/output operations for the system 400. In one implementation, the input/output device 440 includes a keyboard and/or pointing device. In another implementation, the input/output device 440 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system, including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits). The machine learning model can run on Graphic Processing Units (GPUs) or custom machine learning inference accelerator hardware.

To provide for interaction with a user, the features can be implemented on a computer having a display device such as a CRT (cathode ray tube) or LCD (liquid crystal display) monitor for displaying information to the user and a keyboard and a pointing device, such as a mouse or a trackball by which the user can provide input to the computer. Additionally, such activities can be implemented via touchscreen flat panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can, in some cases, be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

The foregoing description is provided in the context of one or more particular implementations. Various modifications, alterations, and permutations of the disclosed implementations can be made without departing from scope of the disclosure. Thus, the present disclosure is not intended to be limited only to the described or illustrated implementations but is to be accorded the widest scope consistent with the principles and features disclosed herein.

In other words, although this disclosure has been described in terms of certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure.

1. A method comprising:
measuring a plurality of n particles, and for each particle i determining:
a particle volume
$${v}_{i}^{p} ;$$
a particle surface area
$${s}_{i}^{p} ;$$
a particle maximum dimension
$${a}_{i}^{p} ;$$
a spherical volume
$${v}_{i}^{s}$$
and spherical surface area
$${s}_{i}^{s}$$
associated with a spine having a diameter equal to the particle maximum dimension
$${a}_{i}^{p} ;$$
and
a residual free surface Sᵢ;
determining, for the plurality of particles, a shape irregularity factor η based on the residual free surface Sᵢ and the particle volume
$${v}_{i}^{p}$$
for all n particles;
determining, from the shape irregularity factor η, a minimum amount of paste for combination with the plurality of n particles to create a workable concrete mixture; and
sending a control signal to a concrete preparation system to cause the concrete preparation system to add at least the minimum amount of paste to a concrete mix comprising the plurality of n particles.

2. The method of claim 1, wherein determining the residual free surface Sᵢ for each particle i comprises calculating
$$( \frac{{s}_{i}^{p}}{{v}_{i}^{p}} - \frac{{s}_{i}^{s}}{{v}_{i}^{s}} ) {v}_{i}^{p} .$$

3. The method of claim 1, wherein determining the shape irregularity factor η comprises calculating
$$\frac{4 π}{3 {( 4 π )}^{\frac{3}{2}}} \frac{{\sum }_{i}^{n} {S}_{i}^{\frac{3}{2}}}{{\sum }_{i}^{n} {v}_{i}^{p}}$$
for all n particles.

4. The method of claim 1, wherein determining the minimum amount of paste for combination with the plurality of n particles comprises calculating (1−ρ)+ρη, where ρ is a packing density of the plurality of n particles.

5. The method of claim 4, wherein the packing density ρ is a ratio of volume occupied by the plurality of n particles to the volume of the n particles.

6. The method of claim 1, wherein the determining the minimum amount of paste to combine with the plurality of n particles comprises applying a correction factor to the shape irregularity factor, the correction factor based on a difference in density between the plurality of n particles and the paste.

7. The method of claim 1, wherein measuring the plurality of n particles comprises scanning each particle with an optical scanner and generating a three-dimensional mesh model for each particle.

8. The method of claim 1, wherein the particle maximum dimension represents the longest straight-line length of the particle i.

9. The method of claim 1, wherein the workable concrete mixture is a concrete mixture with a slump in a range of two to eight inches, wherein slump is measured according to ASTM C143.

10. The method of claim 1, wherein sending the control signal to the concrete preparation system comprises controlling operation of a conveying mechanism to add a volume of paste greater than or equal to the minimum amount, to a mixing vessel of the concrete preparation system.

11. A system comprising:
a concrete preparation system configured to add ingredients to a mixing vessel using a conveying mechanism;
a controller configured to:
measure a plurality of n particles, and for each particle i determine:
a particle volume
$${v}_{i}^{p} ;$$
a particle surface area
$${s}_{i}^{p} ;$$
a particle maximum dimension
$${a}_{i}^{p} ;$$
a spherical volume
$${v}_{i}^{s}$$
and spherical surface area
$${s}_{i}^{s}$$
associated with a sphere having a diameter equal to the particle maximum dimension
$${a}_{i}^{p} ;$$
and
a residual free surface Sᵢ;
determine, for the plurality of particles, a shape irregularity factor η based on the residual free surface Sᵢ and the particle volume
$${v}_{i}^{p}$$
for all n particles;
determine, from the shape irregularity factor η, a minimum amount of paste for combination with the plurality of n particles to create a workable concrete mixture; and
send a control signal to a concrete preparation system to cause the concrete preparation system to add at least the minimum amount of paste to a concrete mix comprising the plurality of n particles.

12. The system of claim 11, wherein the conveying mechanism comprises at least one of, a valve, a conveyor belt, or an auger.

13. The system of claim 11, wherein determining the residual free surface Sᵢ for each particle i comprises calculating
$$( \frac{{s}_{i}^{p}}{{v}_{i}^{p}} - \frac{{s}_{i}^{s}}{{v}_{i}^{s}} ) {v}_{i}^{p} .$$

14. The system of claim 11, wherein determining the shape irregularity factor η comprises calculating
$$\frac{4 π}{3 {( 4 π )}^{\frac{3}{2}}} \frac{{\sum }_{i}^{n} {S}_{i}^{\frac{3}{2}}}{{\sum }_{i}^{n} {v}_{i}^{p}}$$
for all n particles.

15. The system of claim 11, wherein determining the minimum amount of paste for combination with the plurality of n particles comprises calculating (1−ρ)+ρη, where ρ is a packing density of the plurality of n particles.

16. The system of claim 15, wherein the packing density ρ is a ratio of volume occupied by the plurality of n particles to the volume of the n particles.

17. The system of claim 11, wherein the determining the minimum amount of paste to combine with the plurality of n particles comprises applying a correction factor to the shape irregularity factor, the correction factor based on a difference in density between the plurality of n particles and the paste.

18. The system of claim 11, wherein measuring the plurality of n particles comprises scanning each particle with an optical scanner and generating a three-dimensional mesh model for each particle.

19. The system of claim 11, wherein sending the control signal to the concrete preparation system comprises controlling operation of a conveying mechanism to add a volume of paste greater than or equal to the minimum amount, to a mixing vessel of the concrete preparation system.

20-30. (canceled)

31. A non-transitory, computer-readable medium storing one or more instructions executable by a computer system to perform operations comprising:
measuring a plurality of n particles, and for each particle i determining:
a particle volume
$${v}_{i}^{p} ;$$
a particle surface area
$${s}_{i}^{p} ;$$
a particle maximum dimension
$${a}_{i}^{p} ;$$
a spherical volume
$${v}_{i}^{s}$$
and spherical surface area
$${s}_{i}^{s}$$
associated with a sphere having a diameter equal to the particle maximum dimension
$${a}_{i}^{p} ;$$
and
a residual free surface Sᵢ;
determining, for the plurality of particles, a shape irregularity factor η based on the residual free surface Sᵢ and the particle volume
$${v}_{i}^{p}$$
for all n particles;
determining, from the shape irregularity factor η, a minimum amount of paste for combination with the plurality of n particles to create a workable concrete mixture; and
sending a control signal to a concrete preparation system to cause the concrete preparation system to add at least the minimum amount of paste to a concrete mix comprising the plurality of n particles.
